(19) United States
(12) Patent Application Publication
(10) Pub. No.: US 2025/0244303 A1
(43) Pub. Date: Jul. 31, 2025

(54) APPARATUS AND METHOD FOR DETECTING EVAPOTRANSPIRATION

(71) Applicants: The Regents of the University of Michigan, Ann Arbor, MI (US); The Regents of the University of California, Davis, CA (US)

(72) Inventors: Mark A. Burns, Ann Arbor, MI (US); Wen-Chi Lin, Ypsilanti, MI (US); Brian N. Johnson, Ypsilanti, MI (US); Zachary D. Pritchard, Washington, DC (US); Matthew Jenkins, Davis, CA (US); David Block, Davis, CA (US); Konrad Miller, Sugar Lane, TX (US); Shayla Nikzad, Stanford, CA (US); Autumn Mannsfeld, Leuven (BE)

(21) Appl. No.: 18/848,374

(22) PCT Filed: Mar. 23, 2023
(86) PCT No.: PCT/US2023/016093

§ 371 Date: Sep. 18, 2024

Related U.S. Application Data
(60) Provisional application No. 63/322,850, filed on Mar. 23, 2022.

(51) Int. Cl. G01N 33/00; A01G 25/16

(52) U.S. Cl. CPC G01N 33/0098; A01G 25/167

(57) ABSTRACT

A method for detecting plant evapotranspiration includes: detecting, by an evapotranspiration (ET) sensor arranged in a canopy of a plant, a speed of air flowing through the canopy of plant, a temperature of the air and a relative humidity of the air over a plurality of time intervals; calculating, by a processor, an estimated mass the air using at least one of a convective mass transfer (CMT) model, a mass balance (MB) model, or an empirical (EM) model; determining, by the processor, a plant scaling coefficient; integrating, by the processor, the estimated mass flux of the air over the plurality of time intervals to obtain a running sum; and multiplying, by the processor, the running sum by the plant scaling coefficient to determine an estimated evapotranspiration of the plant.

### PRIORITY

This application claims the benefit of U.S. provisional application entitled “Apparatus and Method for Detecting Evapotranspiration,” filed Mar. 23, 2022, and assigned Ser. No. 63/322,850, the entire disclosure of which is hereby expressly incorporated by reference.

### FIELD

The present disclosure relates to an apparatus and a method for predicting evapotranspiration (ET) in plants.

### BACKGROUND

The statements in this section merely provide background information related to the present disclosure and may not constitute prior art.

Most of the world's wine producing regions are affected by seasonal drought conditions. In particular, California, one of the world's largest sources of wine grapes, recently underwent a five-year period of sustained drought conditions. In California, bulk irrigation is the status quo in regions where irrigati

on is necessary to improve grape yield or quality. This means growers water all the grapevines in a given vineyard block with the same amount of water. However, we have discovered that evapotranspiration (ET) rates depend on factors including, but not limited to: soil type, soil water status, and canopy orientation, and that because these variables differ from plant to plant, bulk irrigation may result in the overwatering of some vines.

The traditional method for measuring ET is by a proxy measurement using a weather station over a well-watered lawn, along with correction factors known as crop coefficients, specific for the type of plant being grown nearby. While this method can be effective at estimating regional crop ET, this estimation may be affected by grape variety, viticultural practices, and other factors. This method also cannot estimate ET at the scale of individual plants or a single vine.

Other methods measure ET accurately for vineyards on the order of 3 to 5 acres using an energy balance approach which calculates the amount of water leaving the plants in this area using measurements of small temperature changes and also a method called surface renewal. However, we have discovered that if biometeorological variability is high in the 5-acre plot, this approach may not give the most efficient approach to irrigation. In addition, the surface renewal method cannot detect plant water status below one-acre resolution.

### SUMMARY

The present disclosure has been made to solve the above-mentioned problems occurring in the prior art while advantages achieved by the prior art are maintained intact.

The present disclosure provides a method that measures at a single vine level and is inexpensive and easy enough to use on every vine in a vineyard or, if cost per acre is to be minimized, then a large subset of vines distributed throughout.

In one form of the present disclosure, the method may predict a single plant ET in grapevines using proximal sensing with simple sensors. The method for detecting plant evapotranspiration comprises: detecting, by an evapotranspiration (ET) sensor arranged in a canopy of a plant, a speed of air flowing through the canopy of plant, a temperature of the air and a relative humidity of the air over a plurality of time intervals; calculating, by a processor, an estimated mass flux (\dot{m}ₑ) of the air using at least one of a convective mass transfer (CMT) model, a mass balance (MB) model, or an empirical (EM) model; determining, by the processor, a plant scaling coefficient; integrating, by the processor, the estimated mass flux of the air over the plurality of time intervals to obtain a running sum; and multiplying, by the processor, the running sum by the plant scaling coefficient to determine an estimated evapotranspiration of the plant.

In particular, the estimated mass flux (\dot{m}ₑ) by the CMT model is calculated by:

$${\dot{m}}_{e} = {K}_{m} \cdot ( {P}_{sat} - {P}_{∞} ) ,$$

where, Kₘ is a mass transfer coefficient, Pₛₐₜ is a partial pressure of water in air at a surface, and P_{∞} is a pressure in the air in atmosphere.

In another form, the estimated mass flux (\dot{m}ₑ) of the air by the CMT model is calculated by:

$${\dot{m}}_{e} = {v}_{∞}^{\frac{1}{2}} \cdot {T}^{\frac{11}{12}} \cdot {K}_{cmt} \cdot Δ P ,$$

where, v_{∞} is a bulk velocity of air, T is an air temperature (K) in a canopy of a plant, and ΔP (g·m⁻²) is a difference between a partial pressure of water in the air in a boundary layer of the canopy and a pressure of the air in atmosphere.

Another embodiment of the present disclosure provides a sensor assembly to detect a temperature, a flow rate and a relative humidity of an air. In particular, the sensor assembly comprises: an electrically non-conductive substrate; electrically conductive traces carried by the electrically non-conductive substrate, the electrically conductive traces comprising electrical circuits to sense a temperature, a flow rate and a relative humidity of an air. The electrical circuits comprise: a temperature sensor circuit to determine the temperature of the air; a relative humidity sensor circuit to determine the relative humidity of the air; and a heater circuit to produce a temperature increase. In this embodiment, a dissipation of the temperature increase is a function of the flow rate of the air passing the sensor assembly such that the dissipation is translated into the flow rate of the air.

In another form of the present disclosure, a sensor module comprises: at least one sensor assembly; and a housing formed with at least three slots to which the at least one sensor assembly is inserted. In particular, the sensor assembly comprises: an electrically non-conductive substrate; electrically conductive traces which is carried by the electrically non-conductive substrate and includes electrical circuits to sense a temperature, and a flow rate of an air, wherein the electrical circuits comprise: a temperature sensor circuit configured to determine the temperature of the air; and a heater circuit configured to produce a temperature increase. Here, a dissipation of the temperature increase is a function of the flow rate of the air passing the sensor assembly such that the dissipation is translated into the flow rate of the air.

In other form, the at least one sensor assembly is inserted into a first slot of the three slots and a proximal end of the at least one sensor is exposed to a second slot among the three slots.

In still another form, the housing includes at least two surfaces each formed with at least two slots into which at least two sensor assemblies are respectively inserted into in orthogonal directions to each other, while a proximal end of each of the at least two sensor assemblies is exposed to the air to be sensed.

Further areas of applicability will become apparent from the description provided herein. It should be understood that the description and specific examples are intended for purposes of illustration only and are not intended to limit the scope of the present disclosure.

### DRAWINGS

In order that the disclosure may be well understood, there will now be described various forms thereof, given by way of example, reference being made to the accompanying drawings, in which:

FIG. 1 is a block diagram of an irrigation management system according to one embodiment of the present disclosure;

FIG. 2 is a schematic diagram of the sensing system in one form of the present disclosure;

FIG. 3 is a schematic diagram illustrating a plant assembly integrated with evapotranspiration (ET) sensors operatively deployed in a vineyard in one embodiment of the present disclosure;

FIG. 4 is a flowchart illustrating a process of determining plant evapotranspiration in another embodiment of the present disclosure;

FIG. 5 is a perspective view of the ET sensor in another form of the present disclosure;

FIG. 6 is a schematic diagram illustrating a circuit and sensor assembly of the ET sensor in another embodiment of the present disclosure;

FIG. 7 illustrates a signal processing module to process signals received from respective ET sensors in one embodiment of the present disclosure;

FIG. 8 is a schematic circuit diagram of the signal processing module in FIG. 6;

FIG. 9A is a perspective view of the ET sensor module in another form of the present disclosure;

FIGS. 9B, 9C and 9D are perspective views of a housing of the ET sensor module in some forms of the present disclosure;

FIG. 10 illustrates a perspective view of the ET sensor bonded with external wires for electrical connection to the signal processing module in one form of the present disclosure; and

FIG. 11 is a schematic circuit diagram of the signal processing module in another form of the present disclosure.

The drawings described herein are for illustration purposes only and are not intended to limit the scope of the present disclosure in any way.

### DETAILED DESCRIPTION

The following description is merely exemplary in nature and is not intended to limit the present disclosure, application, or uses. It should be understood that throughout the drawings, corresponding reference numerals indicate like or corresponding parts and features.

As publicly known in the art, some of exemplary forms may be illustrated in the accompanying drawings from the viewpoint of function blocks, units and/or modules. Those having ordinary skill in the art should understand that such blocks, units and/or modules are physically implemented by electronic (or optical) circuits such as logic circuits, discrete components, processors, hard wired circuits, memory devices, and wiring connections. When the blocks, units and or modules are implemented by processors or other similar hardware, the blocks, units and modules may be programmed and controlled through software (for example, codes) in order to perform various functions discussed in the present disclosure. Furthermore, each of the blocks, units and/or modules may be implemented by dedicated hardware or a combination of dedicated hardware for performing some functions and a processor for performing another function (for example, one or more programmed processors and related circuits).

When a component, device, element, or the like of the present disclosure is described as having a purpose or performing an operation, function, or the like, the component, device, or element should be considered herein as being “configured to” meet that purpose or to perform that operation or function.

Although there are currently several means for measuring ET for a single plant or vine, they are expensive enough that only one or a small number of vines can be equipped with a sensor in a vineyard. In addition, the vines equipped with the sensor are chosen to be representative of all the vines in the vineyard and thus this approach is problematic and inaccurate. Aerial imaging and modeling are another viable approach, however, the resolution is dependent on the aerial imaging, and the data processing is not straightforward for most farmers. In other words, these currently used methods are high cost and relatively low resolution.

The present disclosure provides an apparatus and method of sensing evapotranspiration for an irrigation management system that informs watering plants according to individual plants' need and not in bulk, and thus ensuring optimal water use efficiency and improving fruit quality.

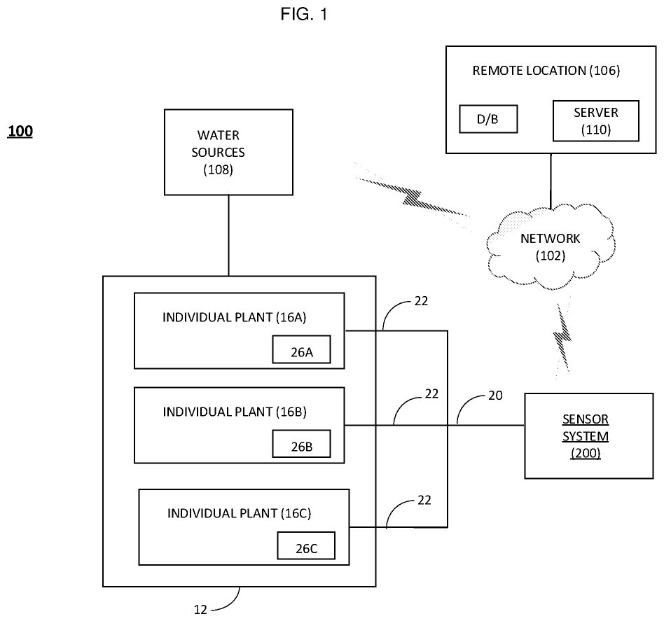

In one form of the present disclosure, FIG. 1 is a block diagram of an irrigation management system in which an illustrative embodiment may be implemented. The irrigation management system 100 includes network 102, which is the medium used to provide communications links between various devices and computers connected together within the irrigation management system 100 such as ET sensors 26A, 26B, and 26C (collectively referred to as ET sensors “26”) which are placed in respective plants 16A, 16B, and 16C (collectively referred to as plants “16”) and connected to a sensor system 200 via cables (20, 22). The cables are connected to a remote location 106. Network 102 may include connections, such as wire, wireless communication links, or fiber optic cables. The remote location 106 may be, for example, personal computers or network computers. The irrigation management system 100 may include additional servers, databases, and other devices that may need to properly manage irrigation of a plurality of plants 16 (e.g., grape vines) in an area 12 (e.g., a grape vineyard). In addition, the irrigation management system 100 includes water resources 108. The water sources 108 are an illustrative example of different sources of water that the irrigation management system 100 can draw upon when providing water to the plurality of plants 16 according to each plant's need as detected by the sensor system 200.

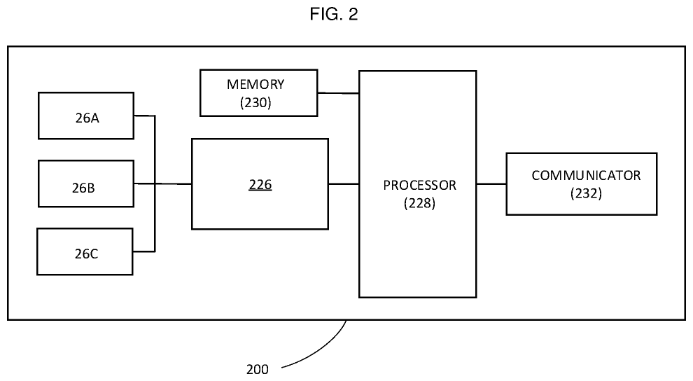

In another form of the present disclosure, the sensor system 200 includes the ET sensors 26 and is configured to measure evapotranspiration (ET) of the individual plants or vines according to the embodiments described below in detail with reference to FIGS. 2-10. The ET of the individual plants is calculated by at least one of three models (i.e., CMT, MB, EM model) described below based on a wind speed (e.g., a speed of air), an air temperature and a relative humidity detected in or near a plant canopy by the ET sensors 26.

FIG. 2 is a schematic diagram of the sensing system 200 in one form of the present disclosure. The sensing system 200 includes: the plurality of ET sensors 26 which are respectively arranged in the plants 16; a signal processing module 226 to process signals received from the respective ET sensors; a memory 230 configured to store data and a set of instructions; a processor 228 configure to execute a program (e.g., the set of instructions); and a communicator 232 configured to communicate with the server 110. The communicator 232 may utilize a communication technology such as wireless Internet, short range communication, and/or mobile communication. The memory 230 is a storage medium such as a RAM, a flash memory, a ROM, an EPROM, an EEPROM, a register, a hard disk, a removable disk, a CD-ROM and the like. In another form, the ET sensors 26 and the signal processing module 226 are implemented in a single unit.

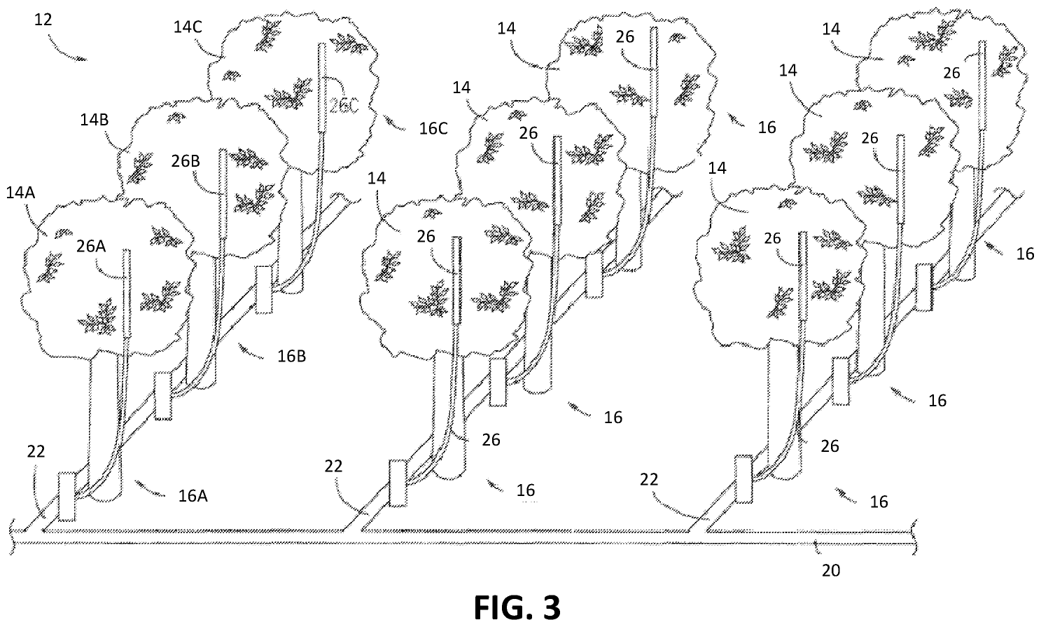

FIG. 3 is a schematic diagram illustrating a plant assembly integrated with evapotranspiration (ET) sensors operatively deployed in a vineyard in one embodiment of the present disclosure. In this embodiment, the ET sensors 26 are a multi-functional sensor which measures a wind speed, an air temperature and a relative humidity in or near the plant canopy 14A, 14B, 14C (collectively referred to as plant canopy “14”).

The measured wind speed, air temperature and relative humidity in or near the plant canopy 14A, 148, 14C are provided to a processor 228 or a remote server 110 via a communicator 232 as illustrated in FIGS. 1-2. In one form, the ET sensors 26 may be implemented in a separate unit from the signal processing module 226 and the processor 228. In another form, the sensor system 200 may be implemented in a single unit integrating the ET sensor 26, the signal processing module 226, the processor 228 and the communicator 232. The processor 228 or the remote server 110 calculates an estimated mass flux (\dot{m}ₑ, units g·s⁻¹ m⁻²) using one of three models (i.e., CMT, MB, and EM models) which are described below in detail.

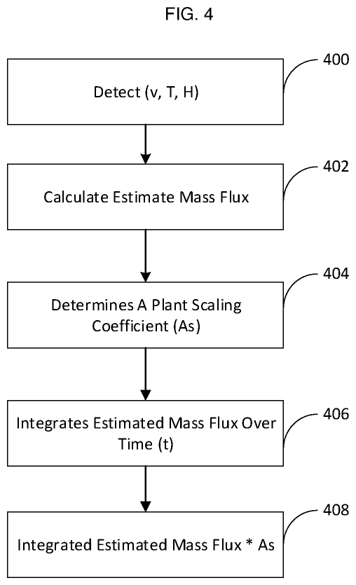

FIG. 4 is a flowchart illustrating a process of a method for calculating an estimated evapotranspiration using the models. The method comprises detecting a speed (v) of air flowing through a canopy 14 of a plant 16, a temperature (T) of the air and a relative humidity (H) of the air over a plurality of time intervals by the ET sensor 26 arranged in the canopy 14 of the plant 16 at Step 400. The ET sensor 26 may be mounted on a branch of the plant 16, near the canopy 14 or inside of the canopy. The location of the ET sensor 26 is not limited thereto, and instead decided based on the type of the plant and the arrangement of the plurality of plants to accurately detect the speed, temperature and relative humidity of the air passing through the canopy.

The method further comprises; calculating, by the processor 228 or the remote server 110, an estimated mass flux (\dot{m}ₑ) using at least one of a convective mass transfer (CMT) model, a mass balance (MB) model, or an empirical (EM) model at Step 402. Then, the processor 228 or the remote server 110 determines a plant scaling coefficient at Step 404; integrates the estimated mass flux (me) over the plurality of time intervals (t) to obtain a running sum at Step 406; and calculates the estimated evapotranspiration (ETₑ) by multiplying the integrated \dot{m}ₑ (i.e., the running sum) by the plant scaling coefficient (Aₛ) at Step 408. In other words, in each model using Equation 1, the estimated mass flux (\dot{m}ₑ) is integrated overtime (t, units s) and multiplied by the plant scaling coefficient (Aₛ, units m²) to obtain the estimated ETₑ.

$${ET}_{e} = {A}_{s} . {\int }_{0}^{t} {\dot{m}}_{e} dt \qquad [ Equation 1 ]$$

where: ETₑ is estimated evapotranspiration of the plant.

In Step 402, the processor 228 or the remote server 110 uses each model (CMT, MB and EM) to calculate three estimated mass flux values, then uses an average of these predictions for the downstream calculation of ET.

The CMT, MB and EM models are respectively described below in detail.

### [Convective Mass Transfer (CMT) Model]

The Convective Mass Transfer (CMT) model is a High Resolution Irrigation (HRI) model and relates transpiration to a theory describing the convective mass transfer from a flat surface of water into moving air. This theory is based on an application of the Reynolds analogy, which suggests a simple relation between different transport phenomena. In this case, the convective heat transfer from a flat solid plate into a fluid with laminar flow over its surface is used as an analogy for the convective mass transfer from a flat surface of liquid or a gas saturated with water vapor into a gas with laminar flow over its surface. From this theory, we have found that the estimated mass transfer flux (\dot{m}ₑ) depends on a mass transfer coefficient (Kₘ) and the difference between partial pressure of water in air at the surface (Pₛₐₜ), and a pressure in the air in greater atmosphere (P_{∞}).

In chemical engineering disciplines, the evaporation of a liquid into a fluid (such as air) flowing over its flat surface is mediated by something called the mass transfer coefficient Kₘ. In the present disclosure, this coefficient Kₘ is defined to be a function of the mass diffusivity of air, the Reynolds number, the Schmidt number and a scalar term. The definitions of the Reynolds number and the Schmidt can be expressed in terms of bulk velocity of air and kinematic viscosity, and in terms of kinematic viscosity and mass diffusivity, respectively. The estimated mass transfer flux (\dot{m}ₑ) in the CMT model is calculated by [Equation 2]:

$${\dot{m}}_{e} = {K}_{m} \cdot ( {P}_{sat} - {P}_{∞} ) ,$$

where, Kₘ is the mass transfer coefficient, Pₛₐₜ is a partial pressure of water in air at the surface, and P_{∞} is a pressure in the air in greater atmosphere. The CMT model requires assuming air is a pure gas, which allows the use of a useful proportionality that has been experimentally verified on numerous occasions, that is that mass diffusivity and kinematic viscosity are proportional to temperature to the 3/2 power (T^{3/2}) and temperature to the 1/2 power (T^{1/2}), respectively. When the pure gas condition is assumed to be true, and all other constants are included in the K_{cmt} term. Including multiple constants in a single constant term, K_{cmt} in this case is a generally acceptable practice in mathematics. Here it is used to reduce the number of terms in the model. The full CMT model can be reduced to [Equation 3]:

$${\dot{m}}_{e} = {v}_{∞}^{\frac{1}{2}} \cdot {T}^{\frac{11}{12}} \cdot {K}_{cmt} \cdot Δ P$$

Where, v_{∞} is a bulk velocity of air (m·s⁻¹), T is an air temperature (K) in the canopy, and ΔP (g·m⁻²) is the difference between partial pressure of water in the air in a boundary layer of the canopy and a pressure of the air in greater atmosphere. A saturation pressure of water in the air (in mmHg) is calculated using Antoine's Equation, which relates a vapor pressure to an air temperature, and the partial pressure is calculated by multiplying this value (i.e., the saturation pressure) by a relative humidity. To adjust units from mmHg to g/cm\hat{ }2, the result is multiplied by a conversation factor of 13595.

This CMT model assumes all abaxial leaf surfaces are saturated with water vapor, perfectly flat and have uniform temperatures equal to the temperature of the air in the canopy. Additionally, stomata are assumed to remain in the open state to maintain constant boundary layer saturation and it is assumed that a laminar flow of wind carries water vapor away from the boundary layer. According to the CMT model, the area term (A) is the total single-sided surface area of the transpiring leaves in the canopy. In other words, the CMT model according to an embodiment of the present disclosure uses a measured area of the leaf area and biometeorological data (windspeed, air temp, and humidity) to model the plant ET.

The MB model to calculate the estimated mass flux (\dot{m}ₑ) is described below in detail.

The MB (Mass Balance) model is based on the concept of mass, which in any closed system mass is constant and is neither created nor destroyed. In the case of a plant canopy, this means the mass flux of water out of the canopy (\dot{m}ₒᵤₜ) is equal to the mass flux of water into the canopy (\dot{m}ᵢₙ) and the estimated mass flux (\dot{m}ₑ) of any evapotranspiration from the plant. With rearrangement, this equation states the estimated mass flux is equal to the difference between the mass flux out of the canopy and mass flux into the canopy, as seen in [Equation 4]: \dot{m}ₑ=\dot{m}ₒᵤₜ−\dot{m}ᵢₙ.

The mass flux may be calculated as a product of the bulk velocity of air (V_{∞}, units m·s⁻¹), a temperature of the air, and a difference in absolute humidity (ΔH, units g·m⁻³). To compute \dot{m}ₑ, the speed of the air, the temperature of the air, and a relative humidity are measured by the ET sensors 26 both outside and inside the plant canopy. The ET sensors 26 (i.e., internal ET sensors) are placed inside of canopy of the plants 16 to measure a speed of the air, a temperature of the air, and a relative humidity of the inside of each canopy. In other words, at least one ET sensor 26 is arranged inside of each canopy of the plants 16. Whereas, in one form of the present disclosure, only one ET sensor 26 (external ET sensor) is placed outside of a large group of the plants 16 to measure a speed of the air, a temperature of the air, and a relative humidity of the air flowing outside of the group of the plants 16, thereby reducing a number of ET sensors and human time to measure the speed of the air, the temperature of the air, and the relative humidity of the plants.

In one form, the measured relative humidity value is converted to an absolute humidity value by a software. The full MB model can be reduced to [Equation 5]:

$${\dot{m}}_{e} = {v}_{∞} \cdot Δ H ,$$

where, ΔH is the difference between an absolute outside humidity value (Hₒᵤₜ) of the air outside the plants and an absolute inside humidity value (Hᵢₙ) of the air inside the plant canopy. Absolute humidity and a function of air temperature are computed using a formula derived from the Ideal Gas Law and an equation for Saturation Vapor Pressure from Rick Snyder. The MB model assumes that the difference between the Hᵢₙ inside the plant canopy and the Hₒᵤₜ outside of the canopy represents the ET of the plant, and that wind speed carries water out of the plant canopy. Based on the flux balance concepts underlying this model, the area term (A) is the cross-sectional area of the plant canopy. The canopy cross-sectional area (A) is determined using an automated process, at several (4-6) points throughout the season, not every day. The estimated mass flux (\dot{m}ₑ) is integrated over time (t, units s) and multiplied by the canopy cross-sectional area (A) to obtain the estimated ETₑ.

As described below in detail, the Empirical Model (EM) uses a wind speed (speed of air) and temperature of the air measured in the plant canopy 14 (on every plant) by the ET sensors 26.

The Empirical Model (EM) was selected using only statistical methods from a set of more than 25 candidate models exploring a mass flux from the plant canopy as an effect of various combinations of measured biometeorological parameters, as well as the interactions of these parameters. Examples of measured biometeorological parameters tested include windspeed, air temperature, relative humidity, absolute humidity and the interactions of these terms. In this case, interaction is defined mathematically as the product of multiplying two terms (such as windspeed and air temperature). The goals of EM model development were for generalizability and dimensional reduction. In this context, dimensional reduction has the added benefit of reducing the number of sensors.

The full EM model (i.e., Equation 6) is used because in addition to achieving reduced dimensionality, it also performed well in terms of ET predictions when compared to other candidate models. Based on the performance criteria of r² and RMSE, the EM model consistently explains more variation in mass flux than other models.

[Equation 6]: \dot{m}ₑ=k₁·v_{∞}+k₂·T+k₃·(v_{∞}·T), where v_{∞} is a speed of the air (e.g., a bulk wind speed), T is a temperature of the air, and k₁, k₂, k₃ are a constant. The constants (k₁, k₂, k₃) may be ignored or assumed to be “1” but included in this model to illustrate that this model was derived from multiple linear modeling techniques. In a simple example, the wind speed and air temperature are measured by the ET sensors 26, then the values of k₁, k₂ and k₃ are assumed to be 1. This EM model assumes that humidity related processes are not strong enough predictors of the mass flux to be included in a model designed to explain variation in the mass flux and inform irrigation decisions.

Each of the models (CMT, MB and EM models) is applicable to any perennial woody plant crop, for example, almonds, other nut trees, citrus, stone fruit, apples, etc. By using one of the models, more accurate mass flux is obtained, and thus more reliable estimated evapotranspiration (ET) of the plant is calculated. Accordingly, the present disclosure enables differential watering in vineyards and orchards according to the ET of each plant, thereby improving the efficiency of water use and also crop quality.

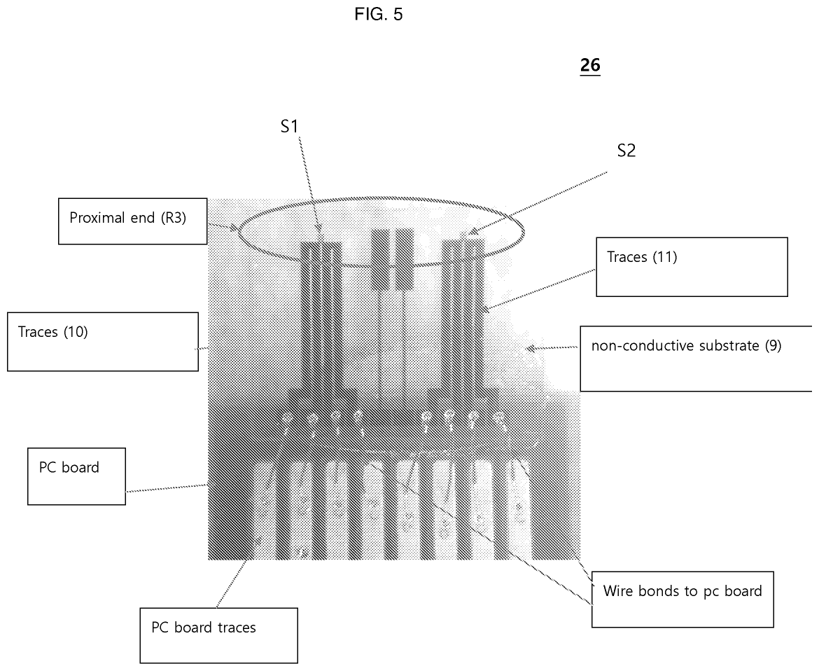

As another form of the present disclosure, the ET sensor 26 (hereinafter “an ET sensor assembly”) and the signal processing module 226 are disclosed below in detail. FIG. 5 is a perspective view of the ET sensor assembly 26 which includes: an electrically non-conductive substrate or a chip body 9, and electrically conductive traces 10, 11 carried by the electrically non-conductive substrate 9. The electrically conductive traces include electrical circuits to sense a temperature, and a flow rate (i.e., a speed) of an air passing through the canopy 14 of the plant. In one embodiment the ET sensor assembly 26 includes a first sensor (S1) having a RTD (Resistance Temperature Detector) element to detect a temperature of air and a second sensor (S2) having another RTD element or a heater element to detect information of the air (e.g., the air flow rate, direction).

The electrical circuits include: a temperature sensor circuit connected to the first sensor S1 to determine the temperature of the air; and a heater circuit connected to the second sensor S2 to produce a temperature increase. In this embodiment, a dissipation of the temperature increase is a function of the flow rate of the air passing the ET sensor assembly 26 such that the dissipation is translated into the flow rate of the air. As shown in FIG. 5, the proximal end R3 is exposed to a medium to be sensed such as the air which flows through slots formed in a housing 50 of the ET sensor 26 and detects the air flow rate (e.g., a wind speed), and air temperature in or near the plant canopy 14. In one form, a humidity sensor is used to detect humidity of the air and may be a circuit or hardware that already exists and is available from commercial sources. Although described in connection with sensors having a resistor or a heating element, the disclosed methods and systems may use additional or alternative sensors. For instance, other sensors that measure air flow via dissipation of a temperature increase may be used.

In one form, the non-conductive substrate 9 (or the chip body) comprises a single piece substrate that is approximately 4.0 mm by 1.0 mm by 0.5 mm or less, and the substrate or chip body 9 is electrically non-conductive such as, but not restricted to, silicon or glass or an organic polymer such as polyimide, PE or PP or PTFE. In addition, the chip body 9 is coated using lithographic technology in patterns with conductive materials such as platinum and titanium and alloys thereof, forming circuits, leads, and pads deposited on an electrical insulating silicon (Si) substrate. The circuits include a temperature sensor circuit, referred to as a RTD (Resistance Temperature Detector), and a heater circuit configured to produce a temperature increase. The circuits also include a plurality of segments. In another form, the substrate 9 is a printed circuit board (PCB) which is electrically connected to the circuits and the proximal end R3 as well as to an external wire to deliver values detected by the proximal ends R3.

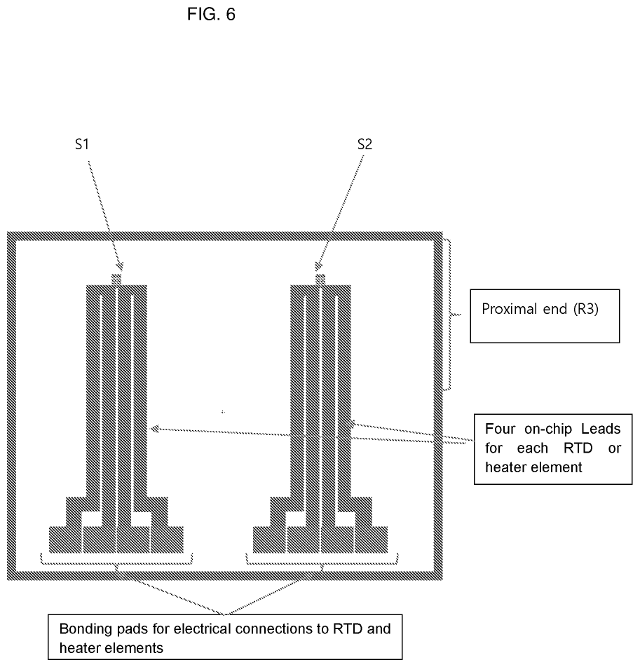

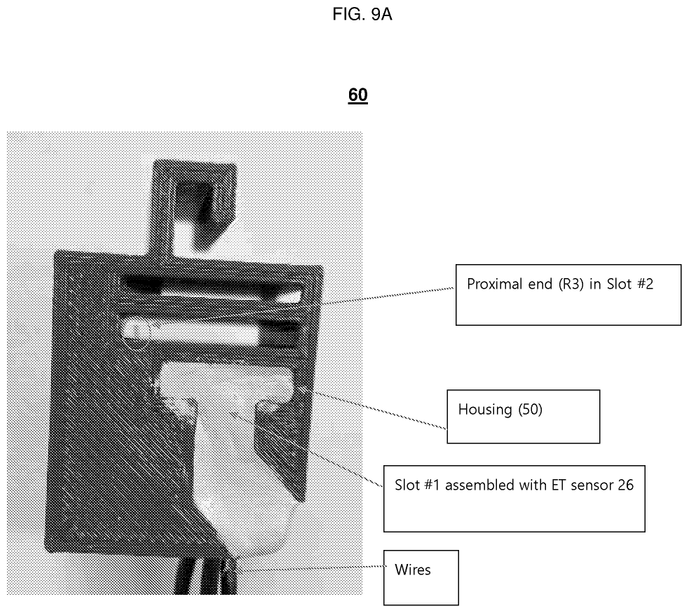

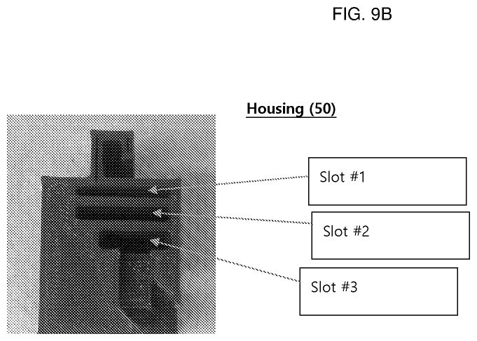

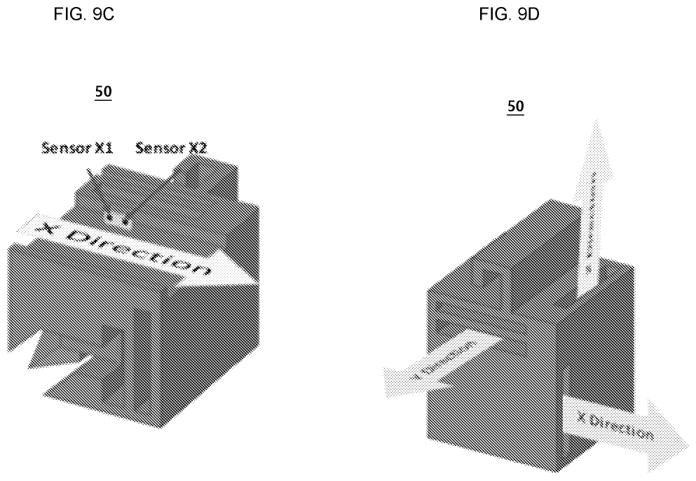

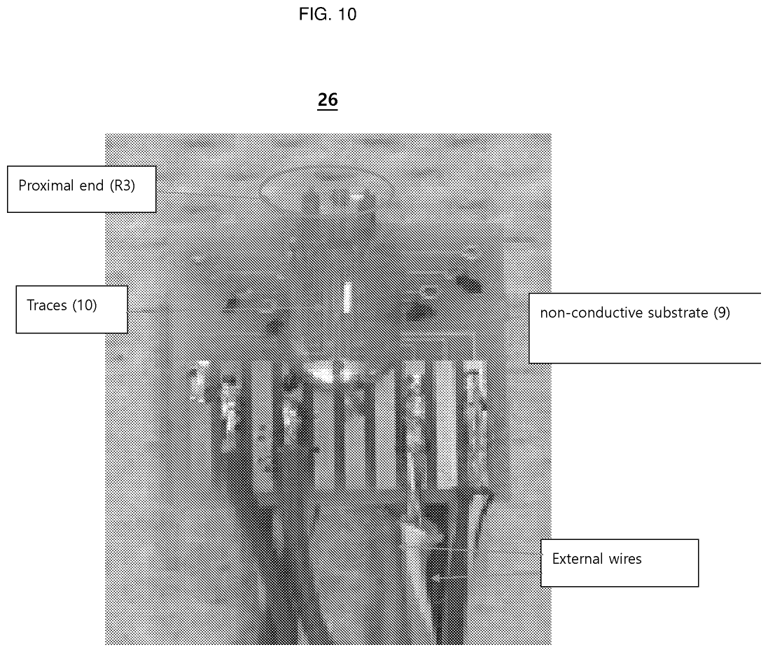

FIG. 6 is a schematic diagram illustrating the circuits of the ET sensor assembly 26, the proximal ends R3, on-chip leads for each RTD, and wire bonding pads for electrical connection to the RTD of the ET sensor assembly 26. As shown in FIG. 5, the wire bonding pads are connected to the PC board via wires. The RTD elements in FIG. 6 are a high-resistance section formed by a long narrow metal trace. Detecting resistance change is used to determine temperature or air flow. In one form, FIG. 6 illustrates four on-chip leads for each of the RTD elements, and two on-chip leads carry current to the RTD element and the remaining two on-chip leads measure a voltage across the RTD element. The four leads to each RTD allow sensing in a four-terminal, or Kelvin configuration. Current flows through one pair of the leads, and the voltage across the RTD is measured using the other pair. See FIG. 8, FIG. 9, and FIG. 10 for further details. In another form, the ET sensor assembly 26 and the signal processing module 226 are implemented in a single unit.

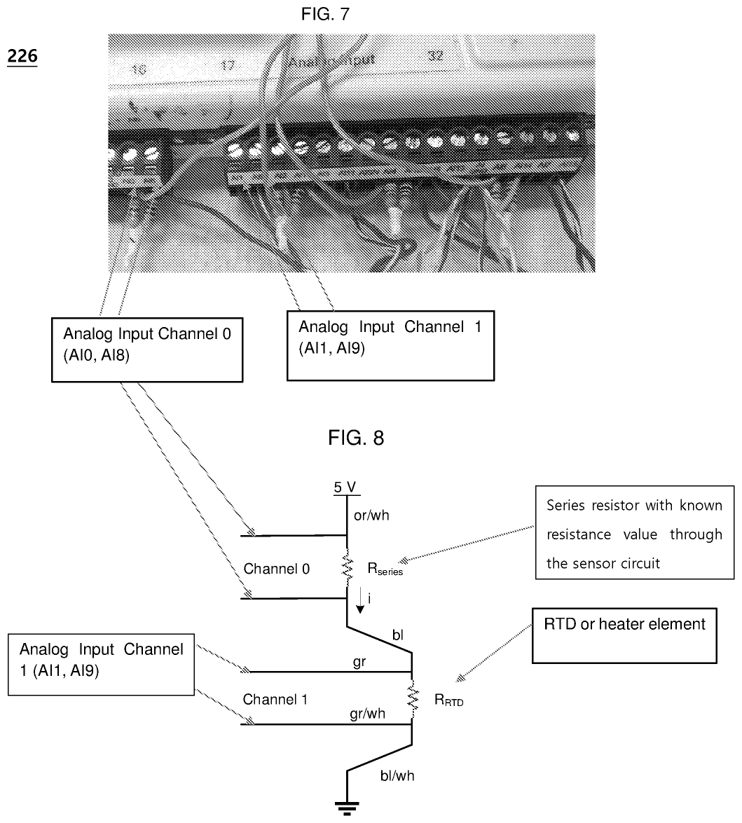

In another form of the present disclosure, FIG. 7 illustrates a partial view of the signal processing module 226 to process signals received from at least one ET sensor assembly 26. For example, the model of the signal processing module 226 may be a USB DAQ module (NI USB-6218) and connected to the ET sensor assembly 26 via connectors (e.g., RJ 45 connectors). Each ET sensor assembly 26 may have two analog input channels operating in differential modes. As illustrated in FIG. 7, the processing module 226 uses a first analog input channel 0 (“AI0”) and a second analog input channel 1 (“AI1”). The differential input configuration of the first analog input channel 0 uses the input “AI0” as positive and “AI8” as negative, and the differential input configuration of the second analog input channel 1 uses the input “AI1” as positive and “AI9” as negative.

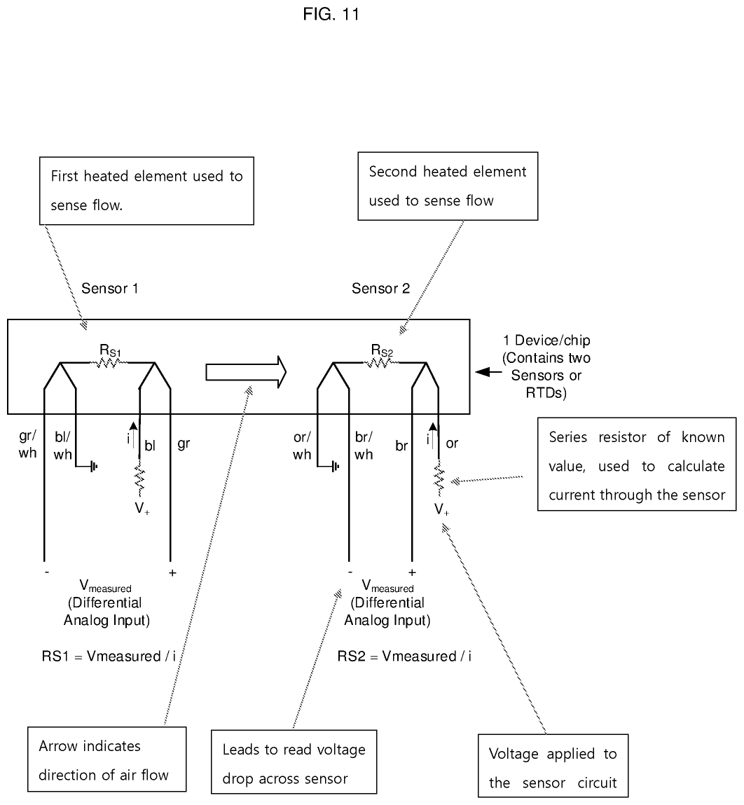

FIG. 8 is a schematic circuit diagram of the signal processing module 226. The illustrated circuit is used to measure a resistance of each RTD element or heater element of the ET sensor assembly 26. FIG. 11 illustrates a circuit diagram with two similar circuits to detect a flow direction of the air based on the different cooling response of each RTD sensor.

In FIG. 8, the current “i” for each ET sensor assembly 26 flows through a series resistor (Rₛₑᵣᵢₑₛ) between a “or/wh” line and a “bl” line, and the voltage drop across this known resistance of the Rₛₑᵣᵢₑₛ is measured by the first differential analog input channel (i.e., Channel 0). The second differential analog input channel (i.e., Channel 1) is used to measure the voltage drop across the sensor itself. In particular, the voltage drop measured across Rₛₑᵣᵢₑₛ by the Channel 0 is used to calculate the current “i”. Using this calculated current and the voltage drop measured across R_{RT}D between the bl line and a “bl/wh” line by the second channel (Channel 1), the resistance of R_{RTD} is calculated and thus the temperature of the air flow is determined.

FIG. 9A is a perspective view of an ET sensor module 60 in another form of the present disclosure, and FIG. 9B is a perspective view of a housing of the ET sensor module 60 in one form of the present disclosure. The ET sensor module 60 includes: at least one ET sensor assembly 26; and the housing 50. The housing 50 is formed with at least three slots, and the ET sensor assembly 26 is inserted in one of the three slots (slot number #1, #2, #3) in a way that the proximal end R3 is exposed into air flow. For example, as illustrated in FIG. 9A, the ET sensor assembly 26 is inserted in the slot #1 and the proximal end R3 is exposed to the slot #2 when assembled to detect the airflow direction (e.g., wind direction).

In another form, two or three ET sensor assemblies 26 may be respectively inserted in the housing 50 in different directions (e.g., x, y and z axis) such that the two or three ET sensor assemblies are arranged to be orthogonal to each other in the housing 50. FIGS. 9C-9D illustrate the configuration of the housing to receive two or three ET sensor assemblies 26 in the different directions. In this embodiment, the housing has a cube shape having surfaces formed with slots to receive the ET sensors while exposing their proximal ends R3 to airflow. FIG. 9C illustrates two proximal ends R3 of the two sensors (X1, X2) are arranged in the x direction and exposed to air flow. FIG. 9D illustrates that the arrangement of three slots to detect air flow in x, y, and z directions. With this specified orthogonal arrangement of the two or three ET sensor assemblies having the proximal ends exposed to the air flow, the ET sensor module 60 may accurately detect the wind direction of the air flow.

FIG. 10 illustrates a perspective view of the ET sensor 26 bonded with external wires for electrical connection to the signal processing module 226. And FIG. 11 illustrates a circuit diagram of the ET sensor assembly 26 having two sensors (S1, S2) each having the RTD element to detect a flow direction of the air based on the different cooling response of each sensor. In FIG. 11, reference characters are used to identify different electrical lines. For example, “gr” refers to a green color line, “or” refers to a orange color line, “br” refers to a brown line, “br/wh” refers to a brown/white color line, “or/wh” refers to a orang/white color line, “or/wh” refers to a orange/white color line, “gr” refers to a green color line, “bl” refers to a blue color line, “bl/wh” refers to a blue/white color line, and “gr/wh” refers to a green/white color line. As shown in FIGS. 5-6 and 10, the two RTD elements of the sensors are placed in a way that they are facing opposite directions and can detect temperature changes in flowing air. In air flow, an upstream sensor (e.g., S1) will experience a larger proportional change in resistance due to cooling than will the downstream sensor (e.g., S2). This change will be measured by the associated electronics as a larger proportional change in voltage on the upstream sensor circuit and a smaller change on the downstream sensor circuit. Knowing the orientation of the two sensors on the chip, the direction of the flow is determined to be from the larger change towards the smaller change.

What is claimed is:

1. A method for detecting plant evapotranspiration, the method comprising:
detecting, by an evapotranspiration (ET) sensor arranged in a plant, a speed of air flowing through the plant, a temperature of the air and a relative humidity of the air over a plurality of time intervals;
calculating, by a processor, an estimated mass flux (\dot{m}ₑ) of the air using at least one of a convective mass transfer (CMT) model, a mass balance (MB) model, or an empirical (EM) model;
determining, by the processor, a plant scaling coefficient;
integrating, by the processor, the estimated mass flux of the air over the plurality of time intervals to obtain a running sum; and
multiplying, by the processor, the running sum by the plant scaling coefficient to determine an estimated evapotranspiration of the plant.

2. The method of claim 1, further comprising:
arranging the ET sensor in a canopy of the plant to detect the speed of air flowing through the canopy of plant, the temperature of the air and the relative humidity of the air over the plurality of time intervals.

3. The method of claim 1, wherein calculating the estimated mass flux (\dot{m}ₑ) of the air includes: calculating the estimated mass flux (\dot{m}e) of the air by the CMT model, and the estimated mass flux (\dot{m}ₑ) is calculated by:
$${\dot{m}}_{e} = {K}_{m} \cdot ( {P}_{sat} - {P}_{∞} ) ,$$
where, Kₘ is a mass transfer coefficient, Pₛₐₜ is a partial pressure of water in air at a surface, and P_{∞} is a pressure in the air in the atmosphere.

4. The method of claim 1, wherein calculating the estimated mass flux (\dot{m}ₑ) of the air includes: calculating the estimated mass flux (\dot{m}ₑ) of the air by the CMT model, and the estimated mass flux (\dot{m}e) is calculated by:
$${\dot{m}}_{e} = {v}_{∞}^{\frac{1}{2}} \cdot {T}^{\frac{11}{12}} \cdot {K}_{cmt} \cdot Δ P ,$$
where, v_{∞} is a bulk velocity of air, T is an air temperature (K) in a canopy of a plant, and ΔP (g·m⁻²) is a difference between a partial pressure of water in the air in a boundary layer of the canopy and a pressure of the air in atmosphere.

5. The method of claim 4, wherein the partial pressure of water in the air in the boundary layer of the canopy is calculated based on a saturation pressure and the relative humidity of the air.

6. The method of claim 1, wherein calculating the estimated mass flux (\dot{m}ₑ) of the air includes: calculating the estimated mass flux (\dot{m}ₑ) of the air by the MB model, and the estimated mass flux (\dot{m}ₑ) is calculated by:
$${\dot{m}}_{e} = {v}_{∞} \cdot Δ H ,$$
where, ΔH is a difference between humidity (Hₒᵤₜ) of the air outside a canopy of the plant and humidity (Hᵢₙ) of the air inside the plant canopy.

7. The method of claim 1, wherein calculating the estimated mass flux (\dot{m}ₑ) of the air includes: calculating the estimated mass flux (\dot{m}ₑ) of the air by the EM model, and the estimated mass flux (\dot{m}ₑ) is calculated by:
$${\dot{m}}_{e} = {k}_{1} \cdot {v}_{∞} + {k}_{2} \cdot T + {k}_{3} \cdot ( {v}_{∞} \cdot T )$$
where, k₁, k₂, k₃ are a constant, v_{∞} is a bulk wind speed, and T is an air temperature (K) in a canopy of a plant.

8. A sensor assembly comprising:
an electrically non-conductive substrate;
electrically conductive traces carried by the electrically non-conductive substrate, the electrically conductive traces comprising electrical circuits to sense a temperature, a flow rate and a relative humidity of an air,
wherein the electrical circuits comprise:
a temperature sensor circuit configured to determine the temperature of the air;
a relative humidity sensor circuit configured to determine the relative humidity of the air; and
a heater circuit configured to produce a temperature increase, and
wherein a dissipation of the temperature increase is a function of the flow rate of the air passing the sensor assembly such that the dissipation is translated into the flow rate of the air.

9. A sensor module comprising:
at least one sensor assembly; and
a housing formed with at least three slots to which the at least one sensor assembly is inserted,
wherein the sensor assembly comprises:
an electrically non-conductive substrate;
electrically conductive traces carried by the electrically non-conductive substrate, the electrically conductive traces comprising electrical circuits to sense a temperature, and a flow rate of an air,
wherein the electrical circuits comprise:
a temperature sensor circuit configured to determine the temperature of the air; and
a heater circuit configured to produce a temperature increase, and
wherein a dissipation of the temperature increase is a function of the flow rate of the air passing the sensor assembly such that the dissipation is translated into the flow rate of the air.

10. The sensor module of claim 9, wherein the at least one sensor assembly is inserted into a first slot of the three slots and a proximal end of the at least one sensor is exposed to a second slot among the three slots.

11. The sensor module of claim 9, wherein the housing includes at least two surfaces each formed with at least two slots into which at least two sensor assemblies are respectively inserted into in orthogonal directions to each other, while a proximal end of each of the at least two sensor assemblies is exposed to the air to be sensed.
